(19) United States
(12) Patent Application Publication
(10) Pub. No.: US 2010/0331558 A1
(43) Pub. Date: Dec. 30, 2010

(54) METHOD FOR PRODUCING FATTY ACID METHYL ESTER

(75) Inventors: Tze-Ming KAO, Taipei City (TW); Chiee-Shyan Lin, Taipei (TW)

Correspondence Address: FRENKEL & ASSOCIATES, 3975 UNIVERSITY DR., STE. 330, FAIRFAX, VA, 22030, US

(73) Assignee: Tze-Ming KAO, Taipei (TW)

(21) Appl. No.: 12/492,332

(22) Filed: Jun. 26, 2009

(51) Int. Cl. C07C 51/00

(52) U.S. Cl. 554/124

(57) ABSTRACT

A method for producing fatty acid methyl ester has steps of providing waste oil with a fatty acid; proceeding a first, second and third esterification; and proceeding transesterification to obtain the fatty acid methyl ester. Because the method of the present invention uses low cost waste oil as a raw material, cost of fatty acid methyl ester can be decreased. Moreover, an amount of free fatty acid in the waste oil can be reduced by esterification, therefore, waste oil used in the transesterification has lowered amount of free fatty acid. Accordingly, the method has increased yield of fatty acid methyl ester.

### BACKGROUND OF THE INVENTION

1. Field of the Invention

The present invention relates to a method for producing fatty acid methyl ester, especially to a method for producing fatty acid methyl ester from inexpensive waste oil with a large amount of free fatty acid including yellow grease, brown grease, black grease or trap grease to save costs.

2. Description of the Prior Arts

Because bio-diesel is burned without producing SOₓ and with decreased amount of polluting gases, bio-diesel such as fatty acid methyl ester can be used as fuel. Generally speaking, there are two methods for producing bio-diesel including esterification and transesterification. Esterification comprises mixing fatty acid and alcohol to form ester and water. Transesterification comprises mixing triglyceride and alcohol to from ester and glycerol.

In transesterification, presence of free fatty acid reduces transesterification yield of triglyceride. Therefore, raw material for transesterification comprises virgin soybean oil and virgin rapeseed oil with less than 0.1 wt % of free fatty acid in order to increase yield of bio-diesel.

However, when the virgin soybean oil or the virgin rapeseed oil is used for producing bio-diesel, raw material costs 60˜75% of production costs of the bio-diesel. Therefore, market price of bio-diesel without favorable government incentives is higher than normal diesel.

Accordingly, for lowering cost, waste frying oils or inedible oils such as yellow grease, brown grease, black grease or trap grease or gutter oil can be used. However, the foregoing oils sold at low cost have a large amount of free fatty acid. Therefore, transesterification yield in transesterification is significantly reduced, so yield of bio-diesel is also decreased.

To overcome the shortcomings, the present invention provides a method for producing fatty acid methyl ester to mitigate or obviate the aforementioned problems.

### SUMMARY OF THE INVENTION

The main objective of the present invention is to provide a method for producing fatty acid methyl ester from inexpensive waste oil with higher percentages of free fatty acid.

A method for producing fatty acid methyl ester in accordance with the present invention has steps of providing waste oil having a fatty acid; first, second and third esterification; and transesterification. Because the method of the present invention uses waste oil as a raw material, cost of fatty acid methyl ester can be decreased. Moreover, an amount of free fatty acid in the waste oil can be reduced by esterification, therefore, waste oil used in the transesterification has lower amounts of free fatty acid. Accordingly, the method has increased yield of fatty acid methyl ester compared to the conventional methods.

Other objectives, advantages and novel features of the invention will become more apparent from the following detailed description when taken in conjunction with the accompanying drawings.

### BRIEF DESCRIPTION OF THE DRAWINGS

FIGS. 1A and 1B is a flow chart of a method for producing fatty acid methyl ester in accordance with the present invention; and

FIGS. 2A and 2B is a flow chart of a method for producing fatty acid methyl ester showing that a second methyl alcohol solution and a fourth methyl alcohol solution can be recycled.

### DETAILED DESCRIPTION OF THE PREFERRED EMBODIMENTS

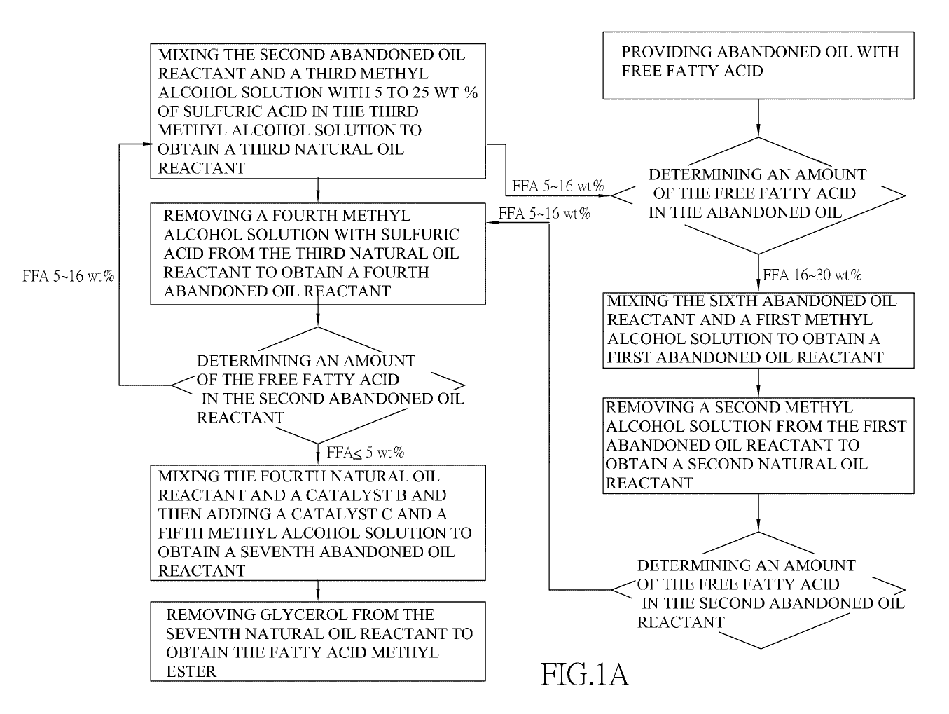

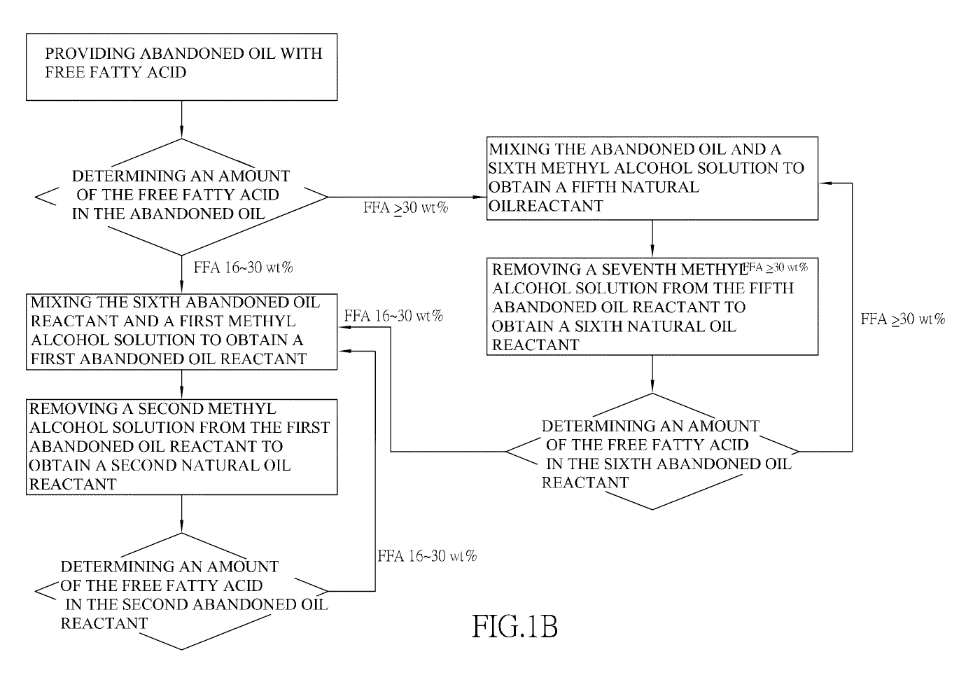

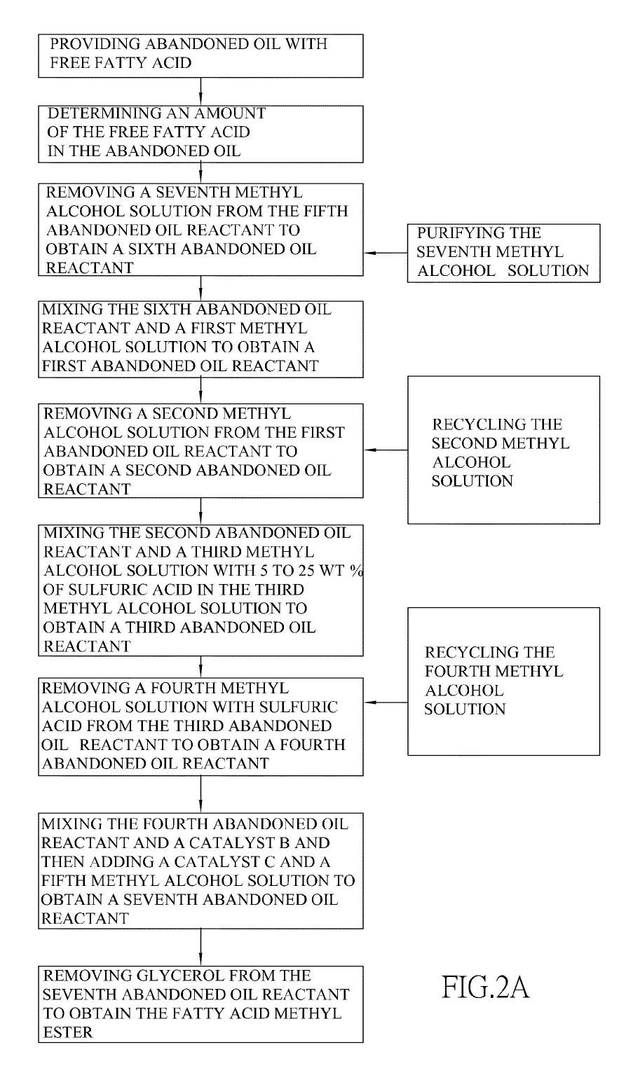

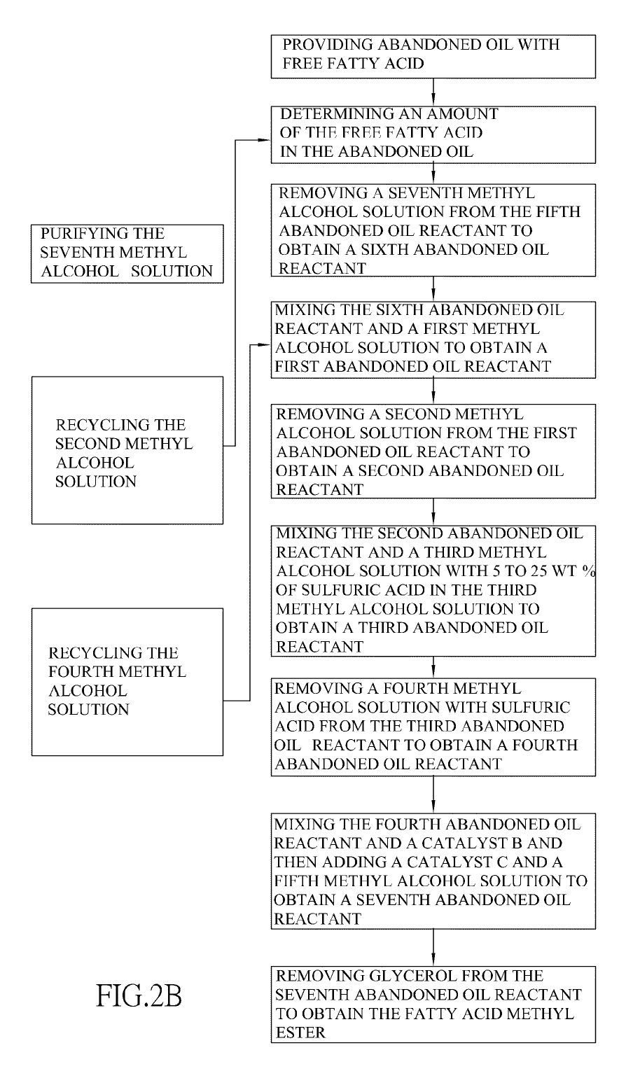

With reference to FIGS. 1 and 2, a method for producing fatty acid methyl ester comprises steps of:

providing waste oil with free fatty acid;

determining an amount of the free fatty acid in the waste oil, wherein when the amount of the free fatty acid is more than 30 wt % in the waste oil, a first esterification is subsequently proceeded; when the amount of the free fatty acid is between 16 and 30 wt % in the waste oil, a second esterification is subsequently proceeded; when the amount of the free fatty acid is between 5 and 16 wt % in the waste oil, a third esterification is subsequently proceeded; wherein

the first esterification has steps of: mixing the waste oil and a sixth methyl alcohol solution to obtain a fifth waste oil reactant; removing a seventh methyl alcohol solution from the fifth waste oil reactant to obtain a sixth waste oil reactant; and determining an amount of free fatty acid in the sixth waste oil reactant; wherein when the amount of the free fatty acid is more than 30 wt % in the sixth waste oil reactant, the first esterification is repeatedly proceeded by recycling the sixth waste oil reactant as the waste oil; when the amount of the free fatty acid is between 16 and 30 wt % in the sixth waste oil reactant, a second esterification is subsequently proceeded;

the second esterification has steps of: mixing the sixth waste oil reactant and a first methyl alcohol solution to obtain a first waste oil reactant; removing a second methyl alcohol solution from the first waste oil reactant to obtain a second waste oil reactant; and determining an amount of the free fatty acid in the second waste oil reactant; wherein when the amount of the free fatty acid is between 16 and 30 wt % in the second waste oil reactant, the second esterification is repeatedly proceeded by recycling the second waste oil reactant as the sixth waste oil reactant; when the free fatty acid is between 5 and 16 wt % in the second waste oil reactant, a third esterification is subsequently proceeded;

the third esterification has steps of: mixing the second waste oil reactant and a third methyl alcohol solution with 5 to 25 wt % of sulfuric acid in the third methyl alcohol solution to obtain a third waste oil reactant; and a molar ratio of the third methyl alcohol solution and free fatty acid in the second waste oil reactant is from 22:1 to 200:1; removing a fourth methyl alcohol solution with sulfuric acid from the third waste oil reactant to obtain a fourth waste oil reactant; and determining the amount of free fatty acid in the fourth waste oil reactant; wherein when the amount of free fatty acid is between 5 and 16 wt % in the fourth waste oil reactant, the third esterification is repeatedly proceeded by recycling the fourth waste oil reactant as the second waste oil reactant; when the free fatty acid is between 0 and 5 wt % in the fourth waste oil reactant, a transesterification is subsequently proceeded; wherein

the transesterification has steps of: mixing the fourth waste oil reactant and a catalyst B and then adding a catalyst C and a fifth methyl alcohol solution to obtain a seventh waste oil reactant; and removing glycerol from the seventh waste oil reactant to obtain the fatty acid methyl ester.

The first methyl alcohol solution, the second methyl alcohol solution and the fourth methyl alcohol solution respectively contain more than 0.3 wt % of water in each methyl alcohol solution. The third methyl alcohol solution and the fifth methyl alcohol solution respectively contain less than 0.3 wt % of water in each methyl alcohol solution.

Preferably, the method for producing fatty acid methyl ester further comprises a step of recycling the second methyl alcohol solution after the step of removing the second methyl alcohol solution from the first waste oil reactant and the second methyl alcohol solution is recycled to serve as a sixth methyl alcohol solution in a first esterification of another method of the present invention.

Preferably, the method for producing fatty acid methyl ester further comprises a step of recycling the fourth methyl alcohol solution after the step of removing the fourth methyl alcohol solution from the first waste oil reactant and the fourth methyl alcohol solution is recycled to serve as a first methyl alcohol solution in a second esterification of another method of the present invention.

Preferably, the method for producing fatty acid methyl ester further comprises a step of purifying the seventh methyl alcohol solution after the step of removing a seventh methyl alcohol solution from the first waste oil reactant and the seventh methyl alcohol solution is recycled to serve as a third methyl alcohol solution in a third esterification or as a fifth methyl alcohol solution of another method of the present invention.

Preferably, the method for producing fatty acid methyl ester further comprises a step of removing impurities from the waste oil after the step of providing waste oil in the first esterification.

Preferably, the method for producing fatty acid methyl ester further comprises a step of purifying the fatty acid methyl ester after the step of transesterification.

More preferably, the step of purifying the fatty acid methyl ester comprises at least one washing step and a drying step to further remove impurities from the fatty acid methyl ester. The washing step comprises washing the fatty acid methyl ester using water. The drying step comprises drying the fatty acid methyl ester under vacuum.

Preferably, the catalyst B is sodium methoxide dissolved in methanol.

More preferably, a weight ratio of the sodium methoxide to the methanol is from 15 to 45 wt %.

Preferably, the catalyst C is sodium hydroxide dissolved in methanol.

More preferably, a weight ratio of the sodium hydroxide to the methanol is from 5 to 25 wt %.

In describing and claiming the present invention, the following terminology will be used in according to the definitions as below.

As used herein, the term “first methyl alcohol solution”, “second methyl alcohol solution”, “fourth methyl alcohol solution”, “sixth methyl alcohol solution” or “seventh methyl alcohol solution” indicates a methanol solution with more than 0.3 wt % of water and may be crude methanol or methanol obtained and recycled from esterification.

As used herein, the term “third methyl alcohol solution” or “fifth methyl alcohol solution” are methanol solution with less than 0.3 wt % of water and may be refined methanol or methanol obtained and recycled from esterification or a transesterification.

### EXAMPLES

The following examples further illustrate the present invention but are not to be construed as limiting the invention as defined in the claims appended hereto.

### Example 1

1120 kg of waste oil with 5.9 wt % of free fatty acid was added to a reaction tank, then was heated to 100° C. and stirred to avoid emulsion formation. The waste oil was kept in the reaction tank for 24 hours to remove impurities from the waste oil. Subsequently, the waste oil was dried at 60° C. under 45 mmHg to remove water from the waste oil. A filter aid was added into the waste oil and the waste oil was filtered to further remove water-soluble impurities from the waste oil and obtain a second waste oil reactant with 5.9 wt % of free fatty acid. Therefore, a second esterification was subsequently conducted.

The second waste oil reactant was mixed with 400 L of a third methanol solution at 62° C. for 3 hours to obtain a third waste oil reactant. The third waste oil reactant was retained for 75 minutes, then a fourth methanol solution and a fourth waste oil reactant were separated by liquid-liquid phase separation. The fourth methanol solution was recycled as a first methanol solution of another first esterification. The fourth waste oil reactant contained 1.58 wt % of free fatty acid. Therefore, a transesterification was subsequently conducted.

The fourth waste oil reactant was mixed with 17.99 kg of catalyst B including methanol and 30 wt % of sodium methoxide dissolved in the methanol to neutralize the fourth waste oil reactant. 2.4 kg of catalyst C including methanol and 15 wt % of sodium hydroxide dissolved in the methanol was then added into the fourth waste oil reactant with catalyst B and mixed for 15 minutes to obtain a seventh waste oil reactant. The seventh waste oil reactant was retained for 2 hours and glycerol and fatty acid methyl ester in the seventh waste oil reactant were separated by liquid-liquid phase separation.

A purification of fatty acid methyl ester was subsequently conducted. The fatty acid methyl ester was sprayed with 500 L water and then was homogeneously mixed with water, kept in water for 2 hours and a water-phase was discharged. Next, the fatty acid methyl ester was sprayed with 500 L water and then was homogeneously mixed with water, kept in water for 20 minutes and a water-phase was discharged. Finally, the fatty acid methyl ester was sprayed with 500 L water and then was homogeneously mixed with water, kept in water for 2 hours and a water-phase was discharged for completely removing impurities from the fatty acid methyl ester. Purified fatty acid methyl ester was dried at 100° C. under 200 mmHg for 1 hour to remove excess water from the purified fatty acid methyl ester.

### Example 2

1196 kg of waste oil with 5.61 wt % of free fatty acid was added to a reaction tank, then was heated to 100° C. and stirred to avoid emulsion formation. Then, the waste oil was kept in the reaction tank for 24 hours to remove impurities from the waste oil. Subsequently, the waste oil was dried at 64° C. under 45 mmHg to remove water form the waste oil. A filter aid was added into the waste oil and the waste oil was filtered to further remove water-soluble impurities form the waste oil and obtain a second waste oil reactant with 5.3 wt % of free fatty acid. Therefore, a second esterification was subsequently conducted.

The second waste oil reactant was mixed with 400 L of a third methanol solution at 56° C. for 45 minutes to obtain a third waste oil reactant. The third waste oil reactant was retained for 75 minutes, then a fourth methanol solution and a fourth waste oil reactant were separated by liquid-liquid phase separation. The fourth methanol solution was recycled as a first methanol solution of another first esterification. The fourth waste oil reactant contained 1.61 wt % of free fatty acid. Therefore, a transesterification was subsequently conducted.

The fourth waste oil reactant was mixed with 16.1 kg of catalyst B including methanol and 30 wt % of sodium methoxide dissolved in the methanol to neutralize the fourth waste oil reactant, 19.2 kg of catalyst C including methanol and 15 wt % of sodium hydroxide dissolved in the methanol was then added into the fourth waste oil reactant with catalyst B and mixed for 15 minutes to obtain a seventh waste oil reactant. The seventh waste oil reactant was retained for 2 hours and glycerol and fatty acid methyl ester in the seventh waste oil reactant were separated by liquid-liquid phase separation.

A purification of fatty acid methyl ester was subsequently conducted, The fatty acid methyl ester was sprayed with 500 L water and then was homogeneously mixed with water, kept in water for 2 hours and a water-phase was discharged. Next, the fatty acid methyl ester was sprayed with 500 L water and then was homogeneously mixed with water, kept in water for 20 minutes and a water-phase was discharged. Finally, the fatty acid methyl ester was sprayed with 500 L water and then was homogeneously mixed with water, kept in water for 2 hours and a water-phase was discharged for completely removing impurities from the fatty acid methyl ester. Purified fatty acid methyl ester was dried at 100° C. under 300 mmHg for 1 hour to remove excess water form the purified fatty acid methyl ester.

### Example 3

1151 kg of waste oil with 51.33 wt % of free fatty acid was added to a reaction tank, then was heated to 100° C. and stirred to avoid emulsion formation. Then, the waste oil was kept in the reaction tank for 24 hours to remove impurities from the waste oil. Subsequently, the waste oil was dried at 70° C. under 70 mmHg to remove water from the waste oil. A filter aid was added into the waste oil and the waste oil was filtered to further remove water-soluble impurities from the waste oil and obtain a sixth waste oil reactant with 50.26 wt % of free fatty acid. Therefore, a first esterification was subsequently conducted.

The first waste oil reactant was mixed with 800 L of a sixth methanol solution at 62° C. for 30 minutes to obtain a fifth waste oil reactant. The fifth waste oil reactant was retained for 75 minutes, then a seventh methanol solution and a sixth waste oil reactant were separated by liquid-liquid phase separation. The seventh methanol solution was recycled by distillation as a third methanol solution of a transesterification. The sixth waste oil reactant contained 29.8 wt % of free fatty acid. Therefore, a second esterification was subsequently conducted.

The sixth waste oil reactant was mixed with 800 L of a first methanol solution at 62° C. for 45 minutes to obtain a first waste oil reactant. The first waste oil reactant was retained for 75 minutes, then a second methanol solution and a second waste oil reactant were separated by liquid-liquid phase separation. The second methanol solution was recycled as a seventh methanol solution of another first esterification. The second waste oil reactant contained 14.1 wt % of free fatty acid. Therefore, a third transesterification was subsequently conducted.

The second waste oil reactant was mixed with 200 L of a first methanol solution at 63° C. for 50 minutes to obtain a third waste oil reactant. The third waste oil reactant was retained for 75 minutes, then a fourth methanol solution and a fourth waste oil reactant were separated by liquid-liquid phase separation. The fourth methanol solution was recycled as a first methanol solution of another second esterification. The second waste oil reactant contained 1.82 wt % of free fatty acid. Therefore, a transesterification was subsequently conducted.

From the first esterification to the third esterification, methanol solutions contained less and less water.

The fourth waste oil reactant was mixed with 10.8 kg of catalyst B including methanol and 30 wt % of sodium methoxide dissolved in the methanol to neutralize the fourth waste oil reactant. 18 kg of catalyst C including methanol and 15 wt % of sodium hydroxide dissolved in the methanol was then added into the fourth waste oil reactant with catalyst B and mixed for 15 minutes to obtain a seventh waste oil reactant. The seventh waste oil reactant was retained for 2 hours and glycerol and fatty acid methyl ester in the seventh waste oil reactant were separated by liquid-liquid phase separation.

A purification of fatty acid methyl ester was subsequently conducted. The free fatty acid methyl ester was sprayed with 500 L water and then was homogeneously mixed with water, kept in water for 2 hours and a water-phase was discharged. Next, the fatty acid methyl ester was sprayed with 500 L water and then was homogeneously mixed with water, retained in water for 1 hour and a water-phase was discharged. Finally, the fatty acid methyl ester was sprayed with 500 L water and then was homogeneously mixed with water, retained in water for 2 hours and a water-phase was discharged for completely removing impurities from the fatty acid methyl ester. Purified fatty acid methyl ester was dried at 100° C. under 200 mmHg for 1 hour to remove excess water from the fatty acid methyl ester.

### Example 4

1117.5 kg of waste oil with 53.87 wt % of free fatty acid was added to a reaction tank, then was heated to 100° C. and stirred to avoid emulsion formation. Then, the waste oil was retained in the reaction tank for 24 hours to remove impurities from the waste oil. Subsequently, the waste oil was dried at 64° C. under 70 mmHg to remove water from the waste oil. A filter aid was added into the waste oil and the waste oil was filtered to further remove water-soluble impurities from the waste oil and obtain a sixth waste oil reactant with 42.24 wt % of free fatty acid. Therefore, a first esterification was subsequently conducted.

The first waste oil reactant was mixed with 800 L of a sixth methanol solution at 63° C. for 15 minutes to obtain a fifth waste oil reactant. The fifth waste oil reactant was retained for 75 minutes, then a seventh methanol solution and a sixth waste oil reactant were separated by liquid-liquid phase separation. The seventh methanol solution was recycled by distillation as a third methanol solution of a transesterification. The sixth waste oil reactant contained 18.3 wt % of free fatty acid. Therefore, a second esterification was subsequently conducted The sixth waste oil reactant was mixed with 800 L of a first methanol solution at 60° C. for 30 minutes to obtain a first waste oil reactant. The first waste oil reactant was retained for 75 minutes, then a second methanol solution and a second waste oil reactant were separated by liquid-liquid phase separation. The second methanol solution was recycled as a seventh methanol solution of another first esterification. The second waste oil reactant contained 14.1 wt % of free fatty acid. Therefore, a third transesterification was subsequently conducted.

The second waste oil reactant was mixed with 400 L of a first methanol solution at 61° C. for 50 minutes to obtain a third waste oil reactant. The third waste oil reactant was retained for 60 minutes, then a fourth methanol solution and a fourth waste oil reactant were separated by liquid-liquid phase separation. The fourth methanol solution was recycled as a first methanol solution of another second esterification. The second waste oil reactant contained 0.82 wt % of free fatty acid. Therefore, transesterification was subsequently conducted.

From the first esterification to the third esterification, methanol solutions contained less and less water.

The fourth waste oil reactant was mixed with 19 kg of catalyst B including methanol and 30 wt % of sodium methoxide dissolved in the methanol to neutralize the fourth waste oil reactant, 8 kg of catalyst C including methanol and 15 wt % of sodium hydroxide dissolved in the methanol was then added into the fourth waste oil reactant with catalyst B and they were mixed for 15 minutes to obtain a seventh waste oil reactant. The seventh waste oil reactant was retained for 2 hours and glycerol and fatty acid methyl ester in the seventh waste oil reactant were separated by liquid-liquid phase separation.

A purification of fatty acid methyl ester was subsequently conducted. The free fatty acid methyl ester was sprayed with 500 L water and then was homogeneously mixed with water, retained in water for 2 hours and a water-phase was discharged. Next, the fatty acid methyl ester was sprayed with 500 L water and then was homogeneously mixed with water, retained in water for 1 hour and a water-phase was discharged. Finally, the fatty acid methyl ester was sprayed with 500 L water and then was homogeneously mixed with water, retained in water for 2 hours and a water-phase was discharged for completely removing impurities from the fatty acid methyl ester. Purified fatty acid methyl ester was dried at 100° C. under 200 mmHg for 1 hour to remove excess water from the fatty acid methyl ester.

Because the method of the present invention uses low cost waste oil as a raw material, cost of fatty acid methyl ester is decreased. Moreover, an amount of free fatty acid in the waste oil can be reduced by esterification, therefore, the waste oil used in the transesterification has lower amounts of free fatty acid. Accordingly, the method has increased yield of fatty acid methyl ester compared to the conventional method.

Even though numerous characteristics and advantages of the present invention have been set forth in the foregoing description, together with details of the structure and function of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A method for producing fatty acid methyl ester, comprising steps of:
providing waste oil with free fatty acid;
determining an amount of the free fatty acid in the waste oil; wherein when the amount of the free fatty acid is more than 30 wt % in the waste oil, a first esterification is subsequently proceeded; when the amount of the free fatty acid is between 16 to 30 wt % in the waste oil, a second esterification is subsequently proceeded; when the amount of the free fatty acid is between 5 to 16 wt % in the waste oil, a third esterification is subsequently proceeded; wherein
the first esterification has steps of:
mixing the waste oil and a sixth methyl alcohol solution to obtain a fifth waste oil reactant;
removing a seventh methyl alcohol solution from the fifth waste oil reactant to obtain a sixth waste oil reactant; and
determining an amount of the free fatty acid in the sixth waste oil reactant; wherein when the amount of the free fatty acid is more than 30 wt % in the sixth waste oil reactant, the first esterification is repeatedly proceeded with the sixth waste oil reactant serving as the waste oil; when the amount of the free fatty acid is between 16 and 30 wt % in the sixth waste oil reactant, the second esterification is subsequently proceeded;
the second esterification has steps of:
mixing the sixth waste oil reactant and a first methyl alcohol solution to obtain a first waste oil reactant;
removing a second methyl alcohol solution from the first waste oil reactant to obtain a second waste oil reactant; and
determining an amount of the free fatty acid in the second waste oil reactant; wherein when the amount of the free fatty acid is between 16 and 30 wt % in the second waste oil reactant, the second esterification is repeatedly proceeded with the second waste oil reactant serving as the sixth waste oil reactant; when the free fatty acid is between 5 and 16 wt % in the second waste oil reactant, a third esterification is subsequently proceeded; and
the third esterification has steps of:
mixing the second waste oil reactant and a third methyl alcohol solution with 5 to 25 wt % of sulfuric acid in the third methyl alcohol solution to obtain a third waste oil reactant; and a molar ratio of the third methyl alcohol solution and free fatty acid in the second waste oil reactant is from 22:1 to 200:1;
removing a fourth methyl alcohol solution with sulfuric acid from the third waste oil reactant to obtain a fourth waste oil reactant; and
determining the amount of free fatty acid in the fourth waste oil reactant; wherein when the amount of free fatty acid is between 5 and 16 wt % in the fourth waste oil reactant, the third esterification is repeatedly proceeded with the fourth waste oil reactant serving as the second waste oil reactant; when the free fatty acid is between 0 to 5 wt % in the fourth waste oil reactant, a transesterification is subsequently proceeded; wherein
the transesterification has steps of:
mixing the fourth waste oil reactant and a catalyst B and then adding a catalyst C and a fifth methyl alcohol solution to obtain a seventh waste oil reactant;
removing glycerol from the seventh waste oil reactant to obtain the fatty acid methyl ester; wherein
the first methyl alcohol solution, the second methyl alcohol solution and the fourth methyl alcohol solution respectively contain more than 0.3 wt % of water in each methyl alcohol solution; and
the third methyl alcohol solution and the fifth methyl alcohol solution respectively contain less than 0.3 wt % of water in each methyl alcohol solution.

2. The method for producing fatty acid methyl ester from fat with high fatty acid as claimed in claim 1 further comprising a step of recycling the second methyl alcohol solution after the step of removing the second methyl alcohol solution from the first waste oil reactant, wherein the second methyl alcohol solution is recycled to serve as a sixth methyl alcohol solution in a first esterification of another method for producing fatty acid methyl ester from fat with high fatty acid.

3. The method for producing fatty acid methyl ester from fat with high fatty acid as claimed in claim 2 further comprising a step of recycling the fourth methyl alcohol solution after the step of removing the fourth methyl alcohol solution from the first waste oil reactant, wherein the fourth methyl alcohol solution is recycled to serve as a first methyl alcohol solution in a second esterification of another method for producing fatty acid methyl ester from fat with high fatty acid.

4. The method for producing fatty acid methyl ester from fat with high fatty acid as claimed in claim 3, the method for producing fatty acid methyl ester further comprises a step of purifying the seventh methyl alcohol solution after the step of removing a seventh methyl alcohol solution from the first waste oil reactant and the seventh methyl alcohol solution is recycled to serve as a third methyl alcohol solution in a third esterification for producing fatty acid methyl ester from fat with high fatty acid.

5. The method for producing fatty acid methyl ester from fat with high fatty acid as claimed in claim 3 further comprising a step of purifying the seventh methyl alcohol solution after the step of removing a seventh methyl alcohol solution from the first waste oil reactant, wherein the seventh methyl alcohol solution is recycled to serve as a fifth methyl alcohol solution of another method for producing fatty acid methyl ester from fat with high fatty acid.

6. The method for producing fatty acid methyl ester from fat with high fatty acid as claimed in claim 4 further comprising a step of removing impurities from the waste oil after the step of providing waste oil in the first esterification.

7. The method for producing fatty acid methyl ester from fat with high fatty acid as claimed in claim 5 further comprising a step of removing impurities from the waste oil after the step of providing waste oil in the first esterification.

8. The method for producing fatty acid methyl ester from fat with high fatty acid as claimed in claim 6 further comprising a step of purifying the fatty acid methyl ester after the step of transesterification.

9. The method for producing fatty acid methyl ester from fat with high fatty acid as claimed in claim 7 further comprising a step of purifying the fatty acid methyl ester after the step of transesterification.

10. The method for producing fatty acid methyl ester from fat with high fatty acid as claimed in claim 8, wherein the step of purifying the fatty acid methyl ester comprises at least one washing step and a drying step to further remove impurities from the fatty acid methyl ester.

11. The method for producing fatty acid methyl ester from fat with high fatty acid as claimed in claim 9, wherein the step of purifying the fatty acid methyl ester comprises at least one washing step and a drying step to further remove impurities from the fatty acid methyl ester.

12. The method for producing fatty acid methyl ester from fat with high fatty acid as claimed in claim 10, wherein the washing step comprises washing the fatty acid methyl ester using water and the drying step comprises drying the fatty acid methyl ester under vacuum.

13. The method for producing fatty acid methyl ester from fat with high fatty acid as claimed in claim 11, wherein the washing step comprises washing the fatty acid methyl ester using water and the drying step comprises drying the fatty acid methyl ester under vacuum.

14. The method for producing fatty acid methyl ester from fat with high fatty acid as claimed in claim 12, wherein the catalyst B has methanol and sodium methoxide dissolved in the methanol.

15. The method for producing fatty acid methyl ester from fat with high fatty acid as claimed in claim 13, wherein the catalyst B has methanol and sodium methoxide dissolved in the methanol.

16. The method for producing fatty acid methyl ester from fat with high fatty acid as claimed in claim 14, wherein a weight ratio of the sodium methoxide and the methanol is from 15 to 45 wt %.

17. The method for producing fatty acid methyl ester from fat with high fatty acid as claimed in claim 15, wherein a weight ratio of the sodium methoxide and the methanol is from 15 to 45 wt %.

18. The method for producing fatty acid methyl ester from fat with high fatty acid as claimed in claim 1, wherein the catalyst C has methanol and sodium hydroxide dissolved in the methanol.

19. The method for producing fatty acid methyl ester from fat with high fatty acid as claimed in claim 18, wherein a weight ratio of the sodium hydroxide and the methanol is from 5 to 25 wt %.
